(19) United States
(12) Patent Application Publication
(10) Pub. No.: US 2026/0033887 A1
(43) Pub. Date: Feb. 5, 2026

(54) BIPOLAR END EFFECTOR ASSEMBLY FOR ROBOTIC SURGICAL INSTRUMENT

(71) Applicant: EndoQuest Robotics, Inc., Houston, TX (US)

(72) Inventors: Byeonghui Kim, Seoul (KR); Jason Seungjoo Lee, Quincy, MA (US); Choyi Kim, Gyeonggi-Do (KR)

(73) Assignee: EndoQuest Robotics, Inc., Houston, TX (US)

(21) Appl. No.: 18/790,627

(22) Filed: Jul. 31, 2024

(51) Int. Cl. A61B 18/14; A61B 18/00; A61B 34/30

(52) U.S. Cl. CPC A61B 18/1445; A61B 34/30; A61B 2018/00077; A61B 2018/00083; A61B 2018/00178; A61B 2018/00184; A61B 2034/306

(57) ABSTRACT

An end effector including a yoke arms, a pivot pin extending between the yoke arms, a pulley mounted on the pivot pin and having a drive pin extending laterally outward from a first side surface thereof, a cam member adjacent the first side surface and including a proximal body having a drive arm, wherein a cam pin is provided at a distal end of the drive arm, and wherein the proximal body has a cam slot that includes a vertical cam path and a horizontal cam path, wherein the vertical cam path is associated with the drive pin and the horizontal cam path is associated with the pivot pin, a first jaw carrier having a cam slot for a first portion of the cam pin and a second jaw carrier having a cam slot for a second portion of the cam pin.

### BACKGROUND OF THE DISCLOSURE

### 1. Field of the Invention

The subject disclosure is directed to surgical instrumentation, and more particularly, to a bipolar end effector assembly for a flexible surgical instrument used in conjunction with a robotic surgical system configured for endoluminal and single-incision surgery.

### 2. Description of Related Art

In robotically assisted minimally invasive surgery, surgical procedures are performed by a surgeon controlling a robotically controlled medical device. The robotically controlled medical device will often have one or more working channels for guiding flexible surgical instruments and videoscopes to an insufflated surgical site. An example of such a robotically controlled medical device is the steerable overtube assembly disclosed in commonly assigned U.S. Pat. No. 11,963,730, the disclosure of which is incorporated herein by reference in its entirety.

Flexible surgical instruments are well known in the art, including for example, the device disclosed in U.S. Pat. No. 10,881,442, which is incorporated herein by reference in its entirety. This instrument includes an end effector having a pair of cooperating jaw members that are operatively connected by of a pivotable coupling link actuated by control wires extending from the proximal end of the instrument. Other flexible surgical instruments of this type are disclosed in commonly assigned U.S. Patent Application No. 2023/0248419, which is incorporated herein by reference in its entirety.

Bipolar surgical instruments are also known in the art and typically include two generally opposing electrodes which are both electrically coupled to an electrosurgical generator and charged to different electric potentials to selectively apply energy to tissue. Bipolar electrosurgical forceps, for example, utilize both mechanical clamping action and electrical energy to treat, e.g., cauterize, coagulate, desiccate, and/or seal, tissue.

The subject disclosure provides an improved flexible surgical instrument for use in endoluminal and/or single-incision robotic surgical procedures, which includes a bipolar end effector assembly having a pair of cooperating jaw members with a unique actuation mechanism designed to convert rotational motion into linear motion.

### SUMMARY OF THE DISCLOSURE

The subject disclosure is directed to a new and useful end effector assembly for a surgical instrument used in endoluminal robotic surgical procedures. The end effector assembly includes a yoke having a proximal body portion defining a longitudinal axis and having first and second spaced apart parallel yoke arms extending distally therefrom to define a gap therebetween. A stationary central pivot pin extends between the parallel yoke arms, perpendicular to the longitudinal axis of the body portion. A pulley is mounted for rotation on the central pivot pin between the parallel yoke arms and it has a drive pin extending laterally outward from a first side surface thereof, facing toward the first yoke arm and located radially outward from the central pivot pin.

The end effector assembly further includes a cam member located adjacent the first side surface of the pulley and including a proximal body portion having an elongated drive arm that extends distally therefrom. A transverse cam pin is provided at a distal end of the drive arm, and the proximal body portion of the cam member has a compound cam slot formed therein. The cam slot includes a vertical cam path and a horizontal cam path, wherein the vertical cam path is associated with the drive pin of the pulley and the horizontal cam path is associated with the central pivot pin of the yoke.

The end effector further includes a first and second jaw carriers. The first jaw carrier has a mounting hub formed on an exterior side surface thereof for supporting a first jaw member and for accommodating the central pivot pin of the yoke. The first jaw carrier also has a first angled cam slot formed in an interior side surface thereof for accommodating a first portion of the cam pin of the cam member. The second jaw carrier has a mounting hub on an exterior side surface thereof for supporting a second jaw member and for accommodating the central pivot pin of the yoke. The second jaw carrier also has a second angled cam slot formed on an interior side surface thereof for accommodating a second portion of the cam pin of the cam member. A first jaw member is supported on the mounting hub of the first jaw carrier and a second jaw member supported on the mounting hub of the second jaw carrier. The first jaw carrier has a first recessed area on the interior side surface thereof for accommodating the cam member and the second jaw carrier has a second recessed area on the interior side surface thereof for accommodating the pulley.

Preferably the end effector is configured as a bipolar device, wherein the first and second jaw members are made of an electrically conductive material, the first and second jaw carriers are made of a non-conductive material, and the yoke is made of a non-conductive material. In addition, a first electrical wire extends through the proximal body portion of the yoke to connect with the first jaw member and a second electrical wire extends through the proximal body portion of the yoke to connect with the second jaw member.

A cable is looped around and secured to an outer periphery of the pulley for rotating the pulley about the central pivot pin to move the first and second jaw members between open and closed positions. The proximal body portion of the yoke is adapted and configured for attachment to a distal end of an instrument shaft and a proximal end of the instrument shaft is operatively connected to an instrument hub that includes a cable actuation mechanism for actuating the cable.

These and other features of the subject disclosure will become more readily apparent to those having ordinary skill in the art to which the subject invention appertains from the detailed description of the preferred embodiments taken in conjunction with the following brief description of the drawings.

### BRIEF DESCRIPTION OF THE DRAWINGS

So that those skilled in the art will readily understand how to make and use the bipolar robotic surgical devices of the subject disclosure without undue experimentation, preferred embodiments thereof will be described in detail herein below with reference to the figures wherein:

FIG. 1 is a perspective view of a robotic surgical system with which the bipolar surgical device of the subject disclosure is employed;

FIG. 2 is a perspective view of the distal end portion of the bipolar surgical device of the subject disclosure;

FIG. 3 is a cross-sectional view taken along lone 3-3 of FIG. 2;

FIG. 4 is a partially exploded perspective view of the end effector assembly of the bipolar surgical device of the subject disclosure;

FIG. 5 is a fully exploded perspective view of the end effector assembly of the bipolar surgical device of the subject disclosure;

FIGS. 6 and 7 are rotated perspective views of the two jaw carriers shown in FIG. 5;

FIG. 8 is a side elevational view of the cam member of the end effector assembly shown in FIG. 5;

FIGS. 9 through 12 are perspective view of jaw members that can be employed in the end effector assembly of the subject disclosure, wherein FIG. 9 is a Cadiere grasper jaw, FIG. 10 is a fenestrated grasper jaw, FIG. 11 is a needle driver jaw, and FIG. 12 is a curved scissor blade;

FIGS. 13 through 15 illustrate movement of the jaw members of the end effector assembly between a closed position and an open position, wherein FIG. 13 shows the jaw members in a closed position, FIG. 14 shown the jaw members in a partially open position, and FIG. 15 shows the jaw member in a fully open position; and

FIGS. 16 and 17 illustrate the proximal actuation assembly for actuating the end effector assembly of the subject disclosure, wherein FIG. 16 corresponds to the jaws moving toward an open between and FIG. 17 corresponds to the jaw members moving toward a closed position.

### DETAILED DESCRIPTION OF THE PREFERRED EMBODIMENTS

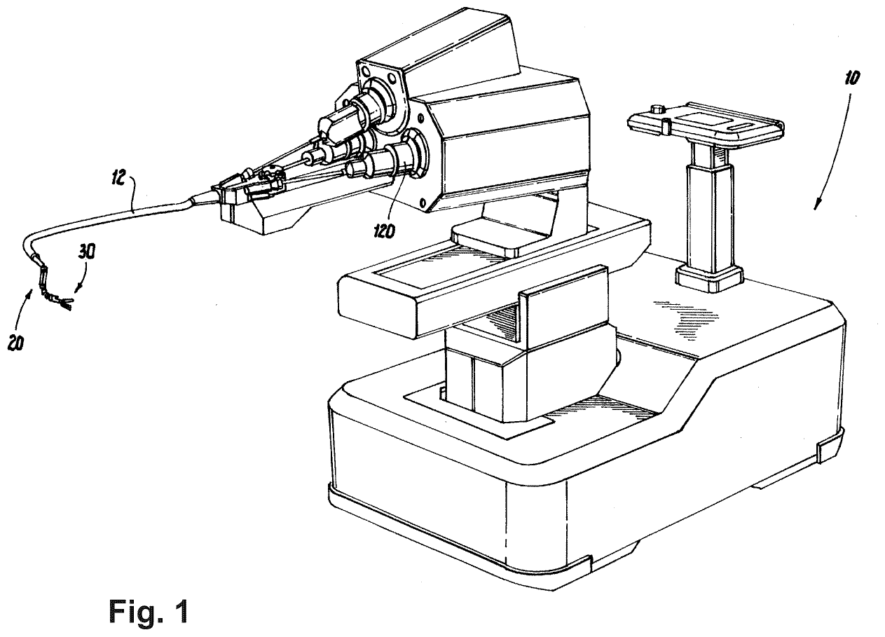

Referring now to the drawings, wherein like reference numerals identify similar structural elements and features of the subject disclosure, there is illustrated in FIG. 1 a patient console for a robotic surgical system with which the flexible surgical instrument of the subject disclosure is used. A patient console of this type, which is designed for use in endoluminal and single-site robotic surgery, is disclosed in commonly assigned U.S. Patent Application Publication 2023/0285098, the disclosure of which is incorporated herein by reference in its entirety.

The robotic surgical system 10 employs a steerable overtube assembly 12 that has a plurality of working channels extending therethrough for accommodating a flexible surgical instrument 20 which includes the bipolar end effector assembly 30 of the subject disclosure. A steerable overtube assembly of this type is disclosed in commonly assigned U.S. Pat. No. 11,963,730, which has been incorporated herein by reference.

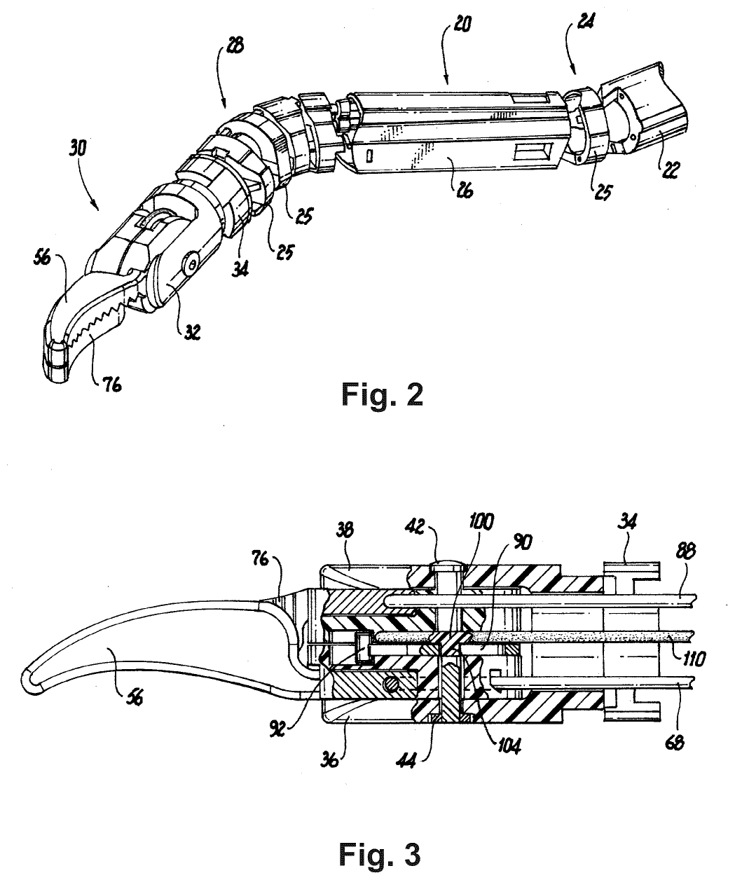

Referring now to FIG. 2, there is illustrated a distal end portion of the flexible surgical instrument 20, which includes an elongated shaft 22, a proximal bendable coupling 24, an elongated medial body portion 26, a distal bendable coupling 28, and a bipolar end effector assembly 30. The proximal bendable coupling 24 connects the medial body portion 26 to the distal end of the elongated shaft 22, and the distal bendable coupling 28 connects the medial body portion to 26 to the bipolar end effector assembly 30. The bendable couplings 24, 28 are constructed from a plurality of operatively connected bending segments 25 of the type disclosed in U.S. Pat. No. 11,504,144, which is incorporated herein by reference in its entirety.

The bipolar end effector assembly 30 is dimensioned for unobstructed travel through the working channels of the overtube assembly 12. More particularly, the end effector assembly preferably has an overall outer diameter of about between 5-6 mm and an overall length, as measured from distal bendable coupling 28, of about between 20-25 mm.

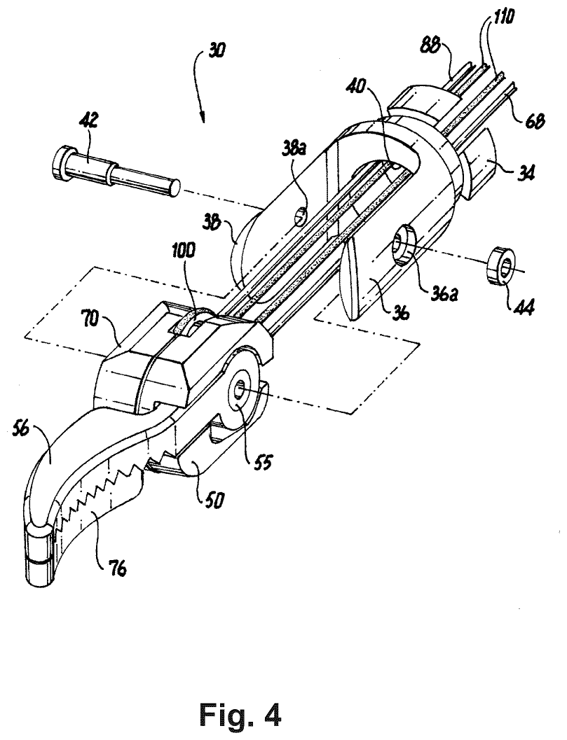

Referring now to FIGS. 3 through 5, the bipolar end effector assembly 30 of the subject disclosure includes a yoke 32 having a proximal body portion 34 defining a longitudinal axis. The yoke 32 further includes first and second spaced apart parallel yoke arms 36 and 38 that extend distally from the proximal body portion 34 to define a gap therebetween. An axial bore 40 extends through the proximal body portion 34 to accommodate control wires and electrical conductors, as discussed in more detail below. A stationary central pivot pin 42 extends between the parallel yoke arms 36 and 38, perpendicular to the longitudinal axis of the body portion 34. A fastener 44 secures the pivot pin 42 in respective apertures 36a and 38a of yoke arms 36 and 38.

The end effector assembly 30 further includes a first and second jaw carriers 50 and 70. The first jaw carrier 50 has an exterior recessed area 52 formed on an exterior side surface 54 thereof for accommodating a first jaw member 56 of the end effector assembly. A first mounting hub 55 is formed within the exterior recessed area 52 for supporting the first jaw member 56 and it has an aperture 58 for accommodating the central pivot pin 42 of yoke 32.

The first jaw carrier 50 also has a first (downwardly) angled cam slot 60 formed in an interior side surface 57 thereof for accommodating a first side portion of a camming pin 92 of cam member 90, which is shown in FIG. 6. The first jaw member 56 has a proximal bushing 62 for receiving the mounting hub 52 of first jaw carrier 50. A connection port 64 is provided in the arm 66 of the first jaw member 56, adjacent the bushing 62, for receiving a first electrical conductor 68, which extends through the central bore 40 of yoke 32 to electrically couple the first jaw 56 member to an external electrosurgical generator (not shown).

As best seen in FIG. 7, the second jaw carrier 70 has an exterior recessed area 72 formed on an exterior side surface 74 thereof for accommodating a second jaw member 76 of the end effector assembly 30. A second mounting hub 75 is formed within the exterior recessed area 72 for supporting the second jaw member 76, and it also has an aperture 78 for accommodating the central pivot pin 42 of yoke 32.

The second jaw carrier 70 has a second (upwardly) angled cam slot 80 formed on an interior side surface 77 thereof for accommodating a second side portion of the camming pin 92 of cam member 90. The inclination of the second angled can slot 80 of jaw carrier 70 is opposite to the inclination of the first angles cam slot 60 of jaw carrier 50. These oppositely angled cam slots facilitates the movement of the opposed movements of the jaw members 56, 76 between open and closed positions, as discussed in more detail below.

The second jaw member 76 has a proximal bushing 82 for receiving the mounting hub 75 of second jaw carrier 70. A port 84 is provided in the arm 86 of the second jaw member 76, adjacent the bushing 82, for receiving a second electrical conductor 88. Electrical conductor 88 extends through the central bore 40 of yoke 32, together with first electrical conductor 68, to electrically couple the second jaw member 76 to an electrosurgical generator.

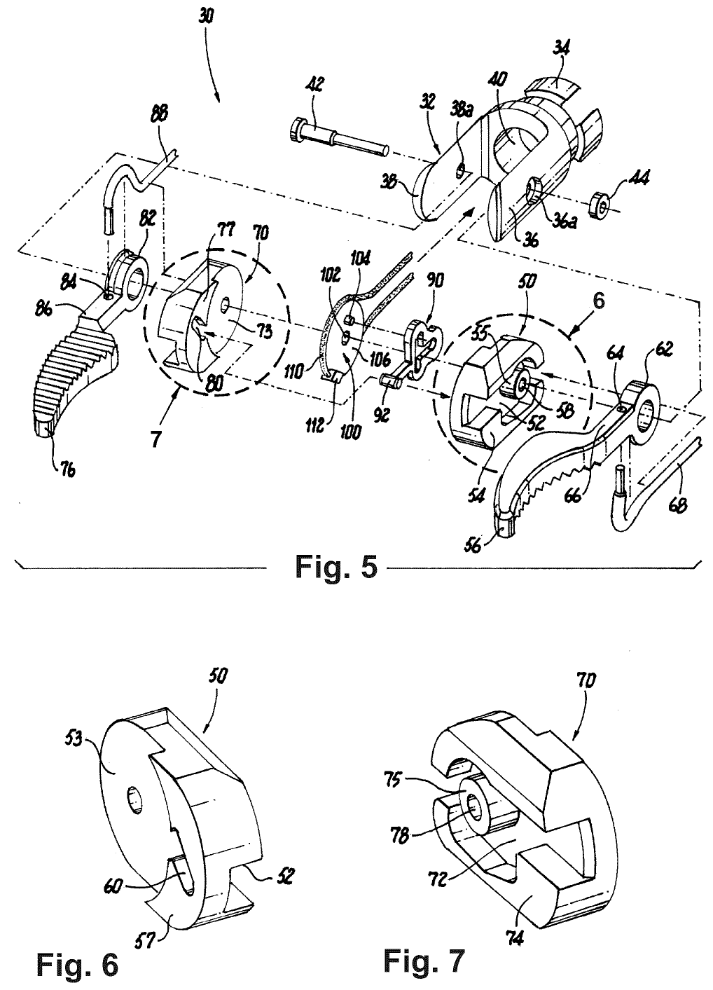

The first jaw carrier 50 also has an interior recessed area 53 on the interior side surface 57 thereof, adjacent the first angled cam slot 60, for accommodating the cam member 90, as best seen in FIG. 6. Similarly, the second jaw carrier 70 has an interior recessed area 73 on the interior side surface 77 thereof, adjacent the second angled cam slot 80 for accommodating a circular actuation pulley 100, which is best seen in FIG. 5. The cam member 90 and the actuation pulley 100 provide the motive forces to open and close the jaw members 56 and 76, as discussed in more detail below.

With continuing reference to FIG. 5, the actuation pulley 100 has a central aperture 102 for rotatably mounting the pulley 100 on the central pivot pin 42. The actuation pulley 100 has a drive pin 104 that extends laterally outward from a first side surface 106 of the pulley 100, facing toward the first jaw carrier 50 and located radially outward from the central aperture 102 of the pulley 100. An actuation cable 110 extends through the central bore 40 of yoke 32 and it is looped around and secured to an outer periphery of the pulley 100 by a clamp 112.

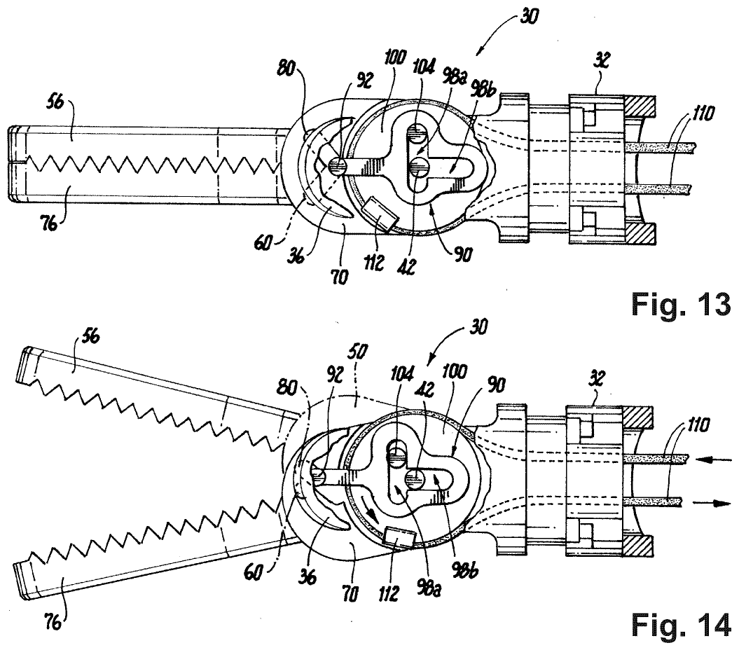

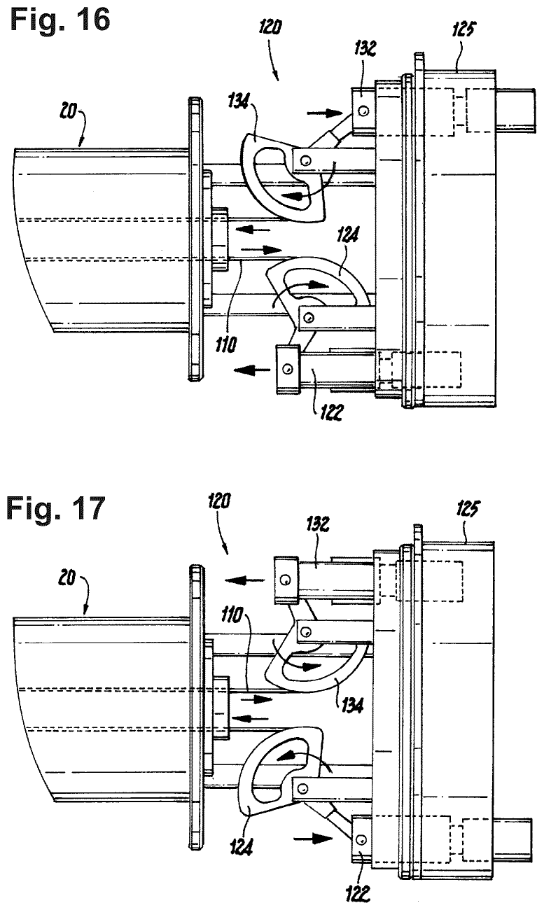

The actuation cable 110 is adapted and configured to rotate the pulley 100 about the central pivot pin 42 so as to move the first and second jaw members 56, 76 between open and closed positions, as discussed in greater detail below with reference to FIGS. 13 through 15. Actuation cable 110 is controlled or otherwise pulled back and forth by an actuation assembly that is operatively associated with a proximal end portion of surgical instrument 20, which will be discussed in greater detail below with reference to FIGS. 16 and 17.

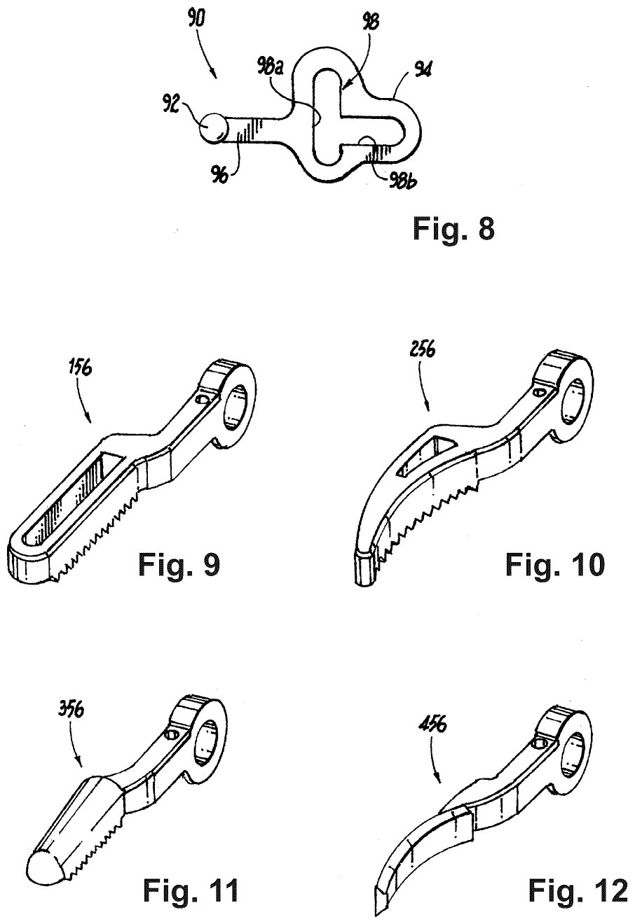

With continuing refence to FIG. 5 in conjunction with FIG. 8, the cam member 90 of end effector assembly 30, which is located adjacent the first side surface 106 of actuation pulley 100, includes a proximal body portion 94 and an elongated drive arm 96 that extends distally from the body portion 94. The transverse camming pin 92, mentioned above, is provided at the distal end of drive arm 96, and it interacts with the oppositely angled cam slots 60 and 80 of the jaw carriers 50 and 70. The proximal body portion 94 of cam member 90 has a compound cam slot 98 formed therein. Cam slot 98 includes a vertical cam path 98a and a horizontal cam path 98b. The vertical cam path 98a is primarily associated with the drive pin 104 of pulley 100 and the horizontal cam path 98b is primarily associated with the central pivot pin 42 of yoke 32.

More particularly, as illustrated in FIGS. 13 through 15, when the cooperating jaw members 56 and 76 of end effector assembly 30 are closed, the transverse camming pin 92 of cam member 90 is in its proximal-most position relative to the angled cam slots 60 and 80, and the drive pin 104 of actuation pulley 100 is vertically aligned with the central pivot pin 42 of yoke 32, as shown in FIG. 13. At such a time, the pulley 100 and the actuation cable 110 are stationary.

To open the cooperating jaw members 56 and 76 of end effector assembly 30, the lower stretch of actuation cable 110 is pulled in a proximal direction, as shown in FIG. 13. This causes the actuation pulley 100 to rotate in a counter-clockwise direction about the central pivot pin 42. In so doing, the drive pin 104 of pulley 100 moves angularly forward along an arcuate path, urging the cam member 90 to move distally in a straight line. Consequently, the transverse coming pin 92 at the end of drive arm 96 moves distal within the oppositely angled cam slots 60 and 80 of jaw carriers 50 and 60, causing the jaw member 56 and 76 to open.

Referring to FIG. 14, as the lower stretch of actuation cable 110 continues to be pulled in a proximal direction, further rotating the actuation pulley in a counter-clockwise direction about the central pivot pin 42, the drive pin 104 of pulley 100 continues to move angularly forward along an arcuate path, urging the cam member 90 to move further distally in a straight line within the oppositely angled cam slots 60 and 80 of jaw carriers 50 and 60. This interaction causes the cooperating jaw member 56 and 76 of end effector assembly 30 to move to their fully open position, shown in FIG. 14. In that fully open position, the jaw members 56 and 76 have an opening angle θ of about 31° and the opening itself is about 8.0 mm wide.

As noted above, the end effector assembly 30 is configured as a bipolar surgical device, wherein the first and second jaw members 56 and 76 are connected to respective electrical conductors 68 and 88. Furthermore, jaw members 56 and 76 are made of an electrically conductive material, such as for example, stainless steel or the like. Still further, the first and second jaw carriers 50 and 70, and the yoke 32 are made of a non-conductive or insulative material, such as a medical-grade plastic material, for example, a polycarbonate material. By constructed the bipolar end effector 30 in this manner, the actuation cable 110 is electrically insulated from the electrified jaw members 56 and 76.

In use, during a robotically-assisted surgical procedure, the two bipolar jaw members 56 and 76 function as opposing electrodes that are charged to different electric potentials to selectively apply energy to tissue. Moreover, being configured as curved forceps, jaw members 56 and 76 utilize both mechanical clamping action and electrical energy to treat, e.g., cauterize, coagulate, desiccate, and/or seal, tissue.

Referring now to FIGS. 16 and 17, there is illustrated the cable actuation assembly 120 having an instrument hub 125 that is operatively associated with the proximal end portion of surgical instrument 20 for controlling or otherwise pulling the actuation cable 110 back and forth to open and close the cooperating jaw members 56 and 76 of end effector assembly 30. (See also FIG. 1). The cable actuation assembly 120 is of the type disclosed in commonly assigned U.S. Patent Application Publication 2023/0255702, the disclosure of which is herein incorporated by refence in its entirety.

Referring to FIG. 16, to open the jaws 56, 76, as shown in FIGS. 13 through 15, the actuator piston 122 in instrument hub 125 is pushed distally, activated by a neodymium magnet. This movement causes the arcuate shaped crank arm 124 to rotate in a clock-wise direction to apply tension on the lower stretch of actuation cable 110 and pull it in a proximal direction. At the same time, actuator piston 132 in instrument hub 125 is pulled proximally, activated by a neodymium magnet. This movement causes the arcuate shaped crank arm 134 to rotate in a clock-wise direction to release tension on the upper stretch of actuation cable 110. Consequently, actuation pulley 100 of end effector assembly 30 rotates in a counter clock-wise direction to drive the camming pin 92 of cam member 90 distally, opening the cooperating jaw members 56 and 76.

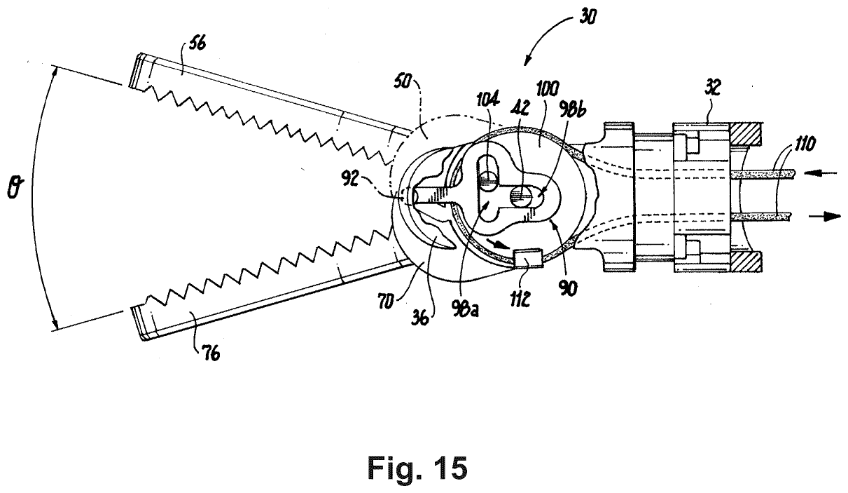

In contrast, FIG. 17 illustrates how the cable actuation assembly 120 functions to move the jaw members 56 and 76 of end effector assembly 30 from the open position of FIG. 15 to the closed position of FIG. 13. More particularly, to close the jaws, actuator piston 122 in instrument hub 125 is pulled proximally, activated by a neodymium magnet. This movement causes the arcuate shaped crank arm 124 to rotate in a counter clock-wise direction to release tension on the lower stretch of actuation cable 110. At the same time, actuator piston 132 in instrument hub 125 is pushed distally, activated by a neodymium magnet. This movement causes the arcuate shaped crank arm 134 to rotate in a counter clock-wise direction to apply tension on the upper stretch of actuation cable 110. Consequently, actuation pulley 100 of end effector assembly 30 rotates in a clock-wise direction to draw the camming pin 92 of cam member 90 proximally, closing the cooperating jaw member 56 and 76.

Referring back to FIGS. 9 through 12, there is illustrated several different embodiments of jaw members that are adapted for use with the bipolar end effector assembly 30 of the subject disclosure. Each of these jaw configurations has a different mechanical function and is designed to be mounted within the jaw carriers 50, 70 described herein, and each one is adapted to be electrically coupled the to an electrosurgical generator by way of an electrical conductor apply electrical energy for treating tissue.

These jaw configurations include, without limitation: the Cadiere-type grasper jaw 156 shown in FIG. 9, which has a jaw length of about 19.0 mm; the fenestrated grasper jaw 256 shown in FIG. 10, which has a jaw length of about 19.0 mm; the needle driver jaw 356 shown in FIG. 11, which has a jaw length of about 8.6 mm; and the curved scissor blade 456 shown in FIG. 12, which has a jaw length of about 11.0 mm. Those skilled in the art will readily appreciate that other jaw types and configurations can be employed herein as well.

While the subject disclosure has been shown and described with reference to preferred embodiments, those skilled in the art will readily appreciate that changes and/or modifications may be made thereto without departing from the scope of the subject disclosure. For example, while the bipolar end effector of the subject disclosure has been described with respect a dual-acting end effector wherein both of the cooperating jaw members move between open and closed positions, it is envisioned and well within the scope of the subject disclosure that the end effector could be configured as a single-acting device, wherein one jaw member is adapted for movement and the other jaw member is stationary. In such an instance, the single-acting end effector jaws could have an open angle of about 25° and an opening width of about 6.4 mm.

What is claimed is:

1. An end effector assembly for a surgical instrument comprising:
a) a yoke having a proximal body portion defining a longitudinal axis and having first and second spaced apart parallel yoke arms extending distally therefrom to define a gap therebetween, wherein a stationary central pivot pin extends between the parallel yoke arms, perpendicular to the longitudinal axis of the body portion;
b) a pulley mounted for rotation on the central pivot pin between the parallel yoke arms and having a drive pin extending laterally outward from a first side surface thereof, facing toward the first yoke arm and located radially outward from the central pivot pin;
c) a cam member adjacent the first side surface of the pulley and including a proximal body portion having an elongated drive arm extending distally therefrom, wherein a transverse cam pin is provided at a distal end of the drive arm, and wherein the proximal body portion of the cam member has a compound cam slot formed therein that includes a vertical cam path and a horizontal cam path, wherein the vertical cam path is associated with the drive pin of the pulley and the horizontal cam path is associated with the central pivot pin of the yoke;
d) a first jaw carrier having a mounting hub formed on an exterior side surface thereof for supporting a first jaw member and for accommodating the central pivot pin of the yoke, the first jaw carrier having a first angled cam slot formed in an interior side surface thereof for accommodating a first portion of the cam pin of the cam member; and
e) a second jaw carrier having a mounting hub on an exterior side surface thereof for supporting a second jaw member and for accommodating the central pivot pin of the yoke, the second jaw carrier having a second angled cam slot formed on an interior side surface thereof for accommodating a second portion of the cam pin of the cam member.

2. An end effector assembly as recited in claim 1, wherein a first jaw member is supported on the mounting hub of the first jaw carrier and a second jaw member supported on the mounting hub of the second jaw carrier.

3. An end effector assembly as recited in claim 1, wherein an actuation cable is looped around and secured to the pulley for rotating the pulley about the central pivot pin to move the first and second jaw members between open and closed positions.

4. An end effector assembly as recited in claim 1, wherein the first jaw carrier has a first recessed area on the interior side surface thereof for accommodating the cam member and the second jaw carrier has a second recessed area on the interior side surface thereof for accommodating the pulley.

5. An end effector assembly as recited in claim 2, wherein the first and second jaw members are made of an electrically conductive material.

6. An end effector assembly as recited in claim 5, wherein the first and second jaw carriers are made of a non-conductive material.

7. An end effector assembly as recited in claim 6, wherein the yoke is made of a non-conductive material.

8. An end effector assembly as recited in claim 7, wherein a first electrical wire extends through the proximal body portion of the yoke to connect with the first jaw member and a second electrical wire extends through the proximal body portion of the yoke to connect with the second jaw member.

9. An end effector assembly as recited in claim 3, wherein the proximal body portion of the yoke is adapted and configured for attachment to a distal end of an instrument shaft.

10. An end effector assembly as recited in claim 9, wherein a proximal end of the instrument shaft is operatively connected to an instrument hub that includes a cable actuation mechanism for actuating the actuation cable.

11. An end effector assembly as recited in claim 3, wherein the actuation cable is clamped to an outer periphery of the pulley.

12. An end effector assembly as recited in claim 2, wherein the first and second jaw members are selected from a group of jaw types consisting of forceps, Cadiere-type graspers, fenestrated graspers, and needle drivers.

13. A surgical instrument comprising:
a) an elongated flexible instrument shaft having opposed proximal and distal end portions;
b) a yoke operatively associated with the distal end portion of the elongated flexible instrument shaft and having a proximal body portion defining a longitudinal axis and having first and second spaced apart parallel yoke arms extending distally therefrom to define a gap therebetween, wherein a stationary central pivot pin extends between the parallel yoke arms, perpendicular to the longitudinal axis of the body portion;
c) a pulley mounted for rotation on the central pivot pin between the parallel yoke arms and having a drive pin extending laterally outward from a first side surface thereof, facing toward the first yoke arm and located radially outward from the central pivot pin;
d) a cam member adjacent the first side surface of the pulley and including a proximal body portion having an elongated drive arm extending distally therefrom, wherein a transverse cam pin is provided at a distal end of the drive arm, and wherein the proximal body portion of the cam member has a compound cam slot formed therein that includes a vertical cam path and a horizontal cam path, wherein the vertical cam path is associated with the drive pin of the pulley and the horizontal cam path is associated with the central pivot pin of the yoke;
e) a first jaw carrier having a mounting hub formed on an exterior side surface thereof for supporting a first jaw member and for accommodating the central pivot pin of the yoke, the first jaw carrier having a first angled cam slot formed in an interior side surface thereof for accommodating a first portion of the cam pin of the cam member;
f) a second jaw carrier having a mounting hub on an exterior side surface thereof for supporting a second jaw member and for accommodating the central pivot pin of the yoke, the second jaw carrier having a second angled cam slot formed on an interior side surface thereof for accommodating a second portion of the cam pin of the cam member;
g) an actuation cable extending through the instrument shaft from the proximal end portion thereof, looped around and secured to the pulley for rotating the pulley about the central pivot pin to move the first and second jaw members between open and closed positions; and
h) an instrument hub operatively associated with the proximal end portion of the instrument shaft and including a cable actuation mechanism for actuating the actuation cable.

14. An end effector assembly as recited in claim 13, wherein a first jaw member is supported on the mounting hub of the first jaw carrier and a second jaw member supported on the mounting hub of the second jaw carrier.

15. An end effector assembly as recited in claim 13, wherein the first jaw carrier has a first recessed area on the interior side surface thereof for accommodating the cam member and the second jaw carrier has a second recessed area on the interior side surface thereof for accommodating the pulley.

16. An end effector assembly as recited in claim 13, wherein the actuation cable is clamped to an outer periphery of the pulley.

17. An end effector assembly as recited in claim 14, wherein the first and second jaw members are made of an electrically conductive material.

18. An end effector assembly as recited in claim 17, wherein the first and second jaw carriers are made of a non-conductive material.

19. An end effector assembly as recited in claim 18, wherein the yoke is made of a non-conductive material.

20. An end effector assembly as recited in claim 19, wherein a first electrical wire extends through the proximal body portion of the yoke to connect with the first jaw member and a second electrical wire extends through the proximal body portion of the yoke to connect with the second jaw member.

21. An end effector assembly as recited in claim 14, wherein the first and second jaw members are selected from a group of jaw types consisting of forceps, Cadiere-type graspers, fenestrated graspers, and needle drivers.
